(19) United States
(12) Patent Application Publication
(10) Pub. No.: US 2026/0192079 A1
(43) Pub. Date: Jul. 9, 2026

(54) METHOD OF ENHANCING MEMORY RECALL THROUGH VR STIMULATION

(71) Applicant: Clarity Technologies SASU, Paris (FR)

(72) Inventors: Carolina Reis, Paris (FR); Raphael Certain, Paris (FR)

(21) Appl. No.: 19/445,378

(22) Filed: Jan. 9, 2026

Related U.S. Application Data
(60) Provisional application No. 63/743,568, filed on Jan. 9, 2025.

(51) Int. Cl. A61M 21/00; G16H 10/60

(52) U.S. Cl. CPC A61M 21/00; G16H 10/60; A61M 2021/0027; A61M 2021/005

(57) ABSTRACT

Systems and methods are disclosed for delivering neuromodulation within immersive virtual-reality environments using personalized and context-rich content. Predefined stimulation protocols are integrated with virtual-reality environments, cognitive tasks, and user-associated multimedia to deliver stimulation in a manner that preserves contextual relevance and realism. By embedding visual, auditory, or multisensory stimulation within ecologically valid virtual experiences, the disclosed systems enable neuromodulation delivery that is more representative of real-world contexts while supporting therapeutic session configuration, execution, and recording.

### CROSS-REFERENCE TO RELATED APPLICATION

This application claims the priority benefit of U.S. provisional patent application No. 63/743,568, filed Jan. 9, 2025, which are incorporated by reference herein in their entirety.

### FIELD OF THE DISCLOSURE

The present disclosure relates to systems and methods for delivering neuromodulation using immersive virtual-reality environments and personalized content to improve the ecological validity of stimulation delivery.

### BACKGROUND

Neurological and neurodegenerative disorders represent a significant and growing global health burden. Such disorders may affect cognitive, emotional, behavioral, or motor functions and often result in progressive impairment that interferes with daily living. Conditions including, but not limited to, Alzheimer's disease, Parkinson's disease, dementia, stroke, traumatic brain injury, and age-related cognitive decline are associated with alterations in neural activity and disruptions of normal brain function. As populations age, the prevalence of these conditions is expected to increase, creating substantial challenges for healthcare systems and caregivers.

Non-invasive neuromodulation techniques have been investigated as potential approaches for influencing neural activity associated with cognitive and functional processes. These techniques may include sensory stimulation delivered through visual, auditory, or multisensory inputs, as well as electrical or magnetic stimulation. Sensory stimulation delivered at specific temporal or spatial parameters has been shown to influence neural oscillations and patterns of synchronized activity in the brain. However, many existing neuromodulation approaches rely on repetitive or abstract stimuli delivered in isolation from meaningful context, which may limit user engagement, adherence, or generalizability to real-world function.

Virtual-reality systems have been explored as tools for delivering immersive experiences that engage users in controlled, interactive environments. Such systems may present simulated scenes, tasks, or activities designed to engage cognitive, emotional, or motor processes. Virtual-reality environments may also incorporate personalized or familiar content, such as representations of places, objects, or experiences meaningful to an individual. While virtual reality has been used for training, rehabilitation, and assessment purposes, its integration with neuromodulation techniques remains limited.

A challenge in existing neuromodulation and stimulation-based therapies is achieving ecological validity, meaning that stimulation is delivered in a manner that reflects realistic, contextually meaningful experiences rather than artificial or disconnected stimuli. Neuromodulation delivered outside of relevant behavioral or cognitive context may fail to engage broader neural networks involved in real-world functioning. There is therefore a need for systems and methods that integrate stimulation protocols with immersive, context-rich environments in a manner that preserves therapeutic intent while enhancing relevance, familiarity, and engagement.

Accordingly, there exists a need for improved systems and methods that combine neuromodulation with immersive virtual-reality environments and personalized content, enabling stimulation to be delivered within meaningful contexts. Such approaches may support more naturalistic engagement with stimulation protocols and provide a flexible framework for therapeutic or intervention-based applications without requiring continuous real-time adaptation or control during session execution.

### SUMMARY OF THE DISCLOSURE

The present disclosure provides systems and methods for delivering neuromodulation within immersive virtual-reality environments using personalized and context-rich content. The disclosed approach addresses limitations of conventional neuromodulation techniques by embedding stimulation delivery within realistic, meaningful, and ecologically valid virtual experiences, rather than presenting stimulation in isolation or abstract settings. In accordance with one or more embodiments, a system includes a performance module configured to assemble and execute virtual-reality-based sessions by selecting virtual-reality environments, cognitive or functional tasks, personalized multimedia, and predefined stimulation protocols from respective data repositories. The assembled session content is delivered to a headset system for presentation to a user. Personalized multimedia associated with the user's history, routines, preferences, or identity may be integrated into the virtual-reality environment and used as contextual anchors for stimulation delivery. Stimulation protocols may define visual, auditory, or multisensory stimulation modalities and associated technical parameters, including stimulation frequency, temporal pattern, spatial pattern, intensity, contrast, or phase relationship. In some embodiments, stimulation is embedded within, overlaid on, or synchronized with elements of the virtual-reality environment or personalized media, enabling neuromodulation delivery in a manner that preserves realism and contextual relevance. Stimulation protocols may be preassociated with a selected session mode or functional domain and remain fixed during session execution. During execution of a session, performance-related and physiological data may be collected by sensors integrated into the headset system and stored without modifying stimulation delivery or session content. A recording module may subsequently retrieve stored data and associate the data with information identifying the virtual-reality environment, cognitive task, personalized multimedia, and stimulation protocol used during the session to generate a session record. The session record may be stored for later review, reporting, or analysis by an authorized user. By integrating predefined neuromodulation protocols with immersive virtual-reality environments and personalized content, the disclosed systems enable delivery of neuromodulation in an ecologically valid manner that more closely reflects real-world contexts while supporting structured session execution and post-session analysis.

### BRIEF DESCRIPTIONS OF THE DRAWINGS

FIG. 1: Illustrates a method of enhancing memory recall through VR stimulation, according to an embodiment.

FIG. 2: Illustrates a Performance Module, according to an embodiment.

FIG. 3: Illustrates a Recording Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

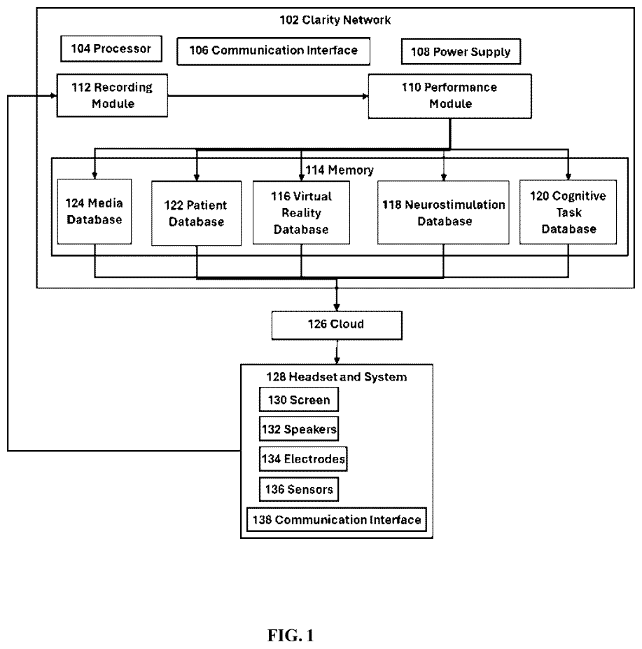

FIG. 1 illustrates a method for enhancing memory recall through VR stimulation. This method comprises a clarity network 102 that integrates patient performance analysis and recording processes with comprehensive databases for cognitive tasks, personalized media, stimulation protocols, and virtual reality environments to deliver immersive and customized memory recall therapy. The clarity network 102 contains a performance module 110 and a recording module 112, which configure therapeutic sessions, analyze patient data, and integrate personalized media with tailored VR environments and stimulation protocols. The clarity network 102 also includes a virtual reality database 116, which stores immersive VR scenarios, the neurostimulation database 118, which provides predefined stimulation protocols aligned with therapeutic goals, the cognitive task database 120, which houses a library of memory, attention, and problem-solving tasks, the media database 124, which stores patient-specific media, and the patient database 122, which maintains patient-specific data, session configurations, and histories. The clarity network 102 connects with the headset 128 to deliver selected virtual-reality environments, personalized multimedia, and stimulation protocols for presentation during a therapeutic session. The clarity network 102 supports reliable data exchange and storage of session information, including performance-related and physiological data collected during session execution, for subsequent review, reporting, or analysis outside the scope of the ongoing session.

Further, embodiments may include a processor 104, also known as a central processing unit (CPU), which may facilitate the operation of the system according to the instructions stored in the memory 114. The processor 104 may also be a graphics processing unit (GPU). The processor 104 may include suitable logic, circuitry, interfaces, and/or code that may be configured to execute a set of instructions stored in the memory 114. The processor 104 may be a hardware component that performs arithmetic, logic, and control operations on data. The processor 104 may be comprised of the arithmetic logic unit, control unit, memory subsystems, and other subsystems. The processor 104 may be responsible for performing arithmetic and logical operations on data. The processor 104 may include components for addition, subtraction, multiplication, and division and logical operations such as AND, OR, and NOT. The processor 104 may be responsible for fetching instructions from memory 114, decoding them, and executing them. The processor 104 may manage the flow of data between different components of the system as a whole, ensuring that operations are performed in the correct order and that data is transferred efficiently. The processor 104 may provide fast access to frequently used data and instructions. The processor 104 may include components such as caches, registers, and pipelines, which are designed to minimize the time required to access and manipulate data. The processor 104 may include various other components and subsystems, such as instruction set architecture (ISA), which may define the set of instructions that the processor 104 can execute. The processor 104 may specify the format of instructions and data, the addressing modes used to access memory 114 and I/O devices, and the interrupt and exception handling mechanisms used to manage errors and other events. The processor 104 may include advanced instruction execution capabilities, support for virtualization and parallel processing, and power management mechanisms that reduce energy consumption and heat dissipation.

Further, embodiments may include a communication interface 106, which may be a hardware or software component that enables communication between two or more electronic devices or systems. The communication interface 106 may include a set of protocols, rules, and standards that define how information is transmitted and received between the devices. The communication interface 106 may be a physical connector, wireless network, or software application. It may include components such as drivers, software libraries, and firmware that may be used to control and manage the communication process. In some embodiments, the communication interface 106 may be compatible with USB, Bluetooth, or Wi-Fi. The communication interface 106 may communicate with a network. Examples of networks may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN).

Further, embodiments may include a power supply 108, which may be an electrical device or system that is used to convert electrical power from a source to a specific form or voltage that an electronic or electrical device can utilize. The power supply 108 may be designed to regulate and control the output power to ensure that the device or system receives the correct amount of power without any damage. The power supply 108 may include a variety of components, such as transformers, rectifiers, filters, voltage regulators, and control circuits that work together to provide the desired output voltage and current. The input power source can be from an AC or DC power source such as a battery, wall outlet, or generator. The power supply 108 may be classified based on various parameters such as the type of output voltage, power rating, efficiency, regulation, and application. In some embodiments, the power supply 108 may include various protection mechanisms such as overvoltage protection, overcurrent protection, short-circuit protection, and thermal protection to ensure safe and reliable operation.

Further, embodiments may include a performance module 110, which manages the configuration and execution of VR-based therapeutic sessions. The performance module 110 facilitates user input via the VR headset 128 interface or a connected system, and assembles session content by selecting and retrieving virtual-reality environments, cognitive tasks, personalized multimedia, and predefined stimulation protocols from respective data repositories. The performance module 110 coordinates delivery of the assembled session content to the headset 128 for presentation to the user. During session execution, the performance module 110 may receive and store performance-related and physiological data generated by the headset system for subsequent review or processing outside the scope of the ongoing session. Further, embodiments may include a recording module 112, which retrieves performance-related and physiological data stored during execution of one or more virtual-reality-based therapeutic sessions. The recording module 112 may associate such data with information identifying the virtual-reality environment, cognitive task, personalized multimedia, and stimulation protocol applied during a session to generate a session record. The recording module 112 may store the session record in one or more data repositories for subsequent review, reporting, or analysis by an authorized user outside the scope of session execution, without modifying stimulation delivery or virtual-reality presentation during the session.

Further, embodiments may include a memory 114, which may store data collected by the clarity network 102, such as sensor data, analysis of data, etc. In one embodiment, the memory 114 may include suitable logic, circuitry, and/or interfaces that may be configured to store a machine code and/or a computer program with at least one code section executable by the processor 104. Examples of implementation of the memory 114 may include, but are not limited to, Random Access Memory (RAM), Read Only Memory (ROM), Hard Disk Drive (HDD), and/or a Secure Digital (SD) card.

Further, embodiments may include a virtual reality database 118, which stores, manages, and delivers immersive virtual reality scenarios and environments for therapeutic applications. The virtual reality database 118 may contain pre-configured and customizable VR content that supports a wide range of cognitive, emotional, and motor therapies. In some embodiments, the virtual reality database 118 may include scenarios such as calming natural landscapes, daily life environments, gamified tasks, cognitive challenges, and personalized content like familiar photographs or memory-based scenes that are tailored to enhance engagement and therapy effectiveness. The virtual reality database 118 contains a collection of 3D environments, animations, sounds, and interactive tasks. In some embodiments, the scenarios may be optimized for seamless integration with the headset and system 128 to ensure smooth rendering and real-time responsiveness. In some embodiments, the virtual reality database 118 may support customization and selection of scenarios based on stored user preferences, session configuration parameters, or predefined session structure. In some embodiments, environmental parameters such as brightness, colors, audio settings, or difficulty levels may be configured prior to session execution based on session settings.

In some embodiments, environmental parameters such as brightness, colors, audio settings, or difficulty levels may be configured prior to session execution based on session settings, user preferences, or predefined session structure. In some embodiments, the virtual reality database 118 may incorporate personalization capabilities, enabling users or caregivers to upload custom content, such as family photos or familiar locations, which can then be synthesized into immersive VR experiences using advanced rendering techniques. In some embodiments, the personalization feature enhances emotional connection and motivation during therapy, ultimately enhancing its efficacy and adherence. In some embodiments, the virtual reality database 118 may be integrated with the system's sensory stimulation protocols, allowing for synchronized delivery of visual and auditory stimuli within the VR environment. For example, flickering objects or modulated sounds at therapeutic frequencies can be seamlessly embedded in a gamified task or relaxation scenario. Visual and auditory stimulation may also be delivered in isolation, such as just a visual flicker and non-modulated audio, and vice versa. In some embodiments, the virtual reality database 118 may store scenarios for meditation, scenarios for relaxation, and scenarios for breathing. In some embodiments, the virtual reality database 118 may contain VR scenes, animated images, sounds, neutral or emotionally charged images or videos, music, cognitive tasks and motor tasks, and scenarios for games. The cognitive tasks and motor tasks may require active participation of the patient during the virtual reality immersion.

Further, embodiments may include a neurostimulation database 118, which may contain instructions for providing visual stimulation protocols, auditory stimulation protocols, and combined audio-visual stimulation protocols. The neurostimulation database 118 may store instructions for providing peripheral nerve stimulation protocols and deep brain stimulation protocols. For example, the neurostimulation database 118 may contain instructions for providing vagus nerve stimulation, transcranial direct current stimulation (tDCS), and transcranial alternating current stimulation (tACS). In some embodiments, stimulations can be provided as rhythmic stimulations. In some embodiments, the stimulation modalities may be delivered daily or according to a different schedule for a given duration. In some embodiments, the duration may be determined depending on factors like whether stimulation is active, such as provided during an assigned task, or passive, such as not provided during an assigned task, and the underlying health status of the patient. The neurostimulation database 118 may be designed to deliver targeted electrical or sensory stimulation to influence neural activity and support therapeutic outcomes. In some embodiments, the neurostimulation database 118 may integrate multiple stimulation modalities, such as non-invasive brain stimulation techniques, including transcranial direct current stimulation (tDCS), transcranial alternating current stimulation (tACS), and sensory stimulations like visual and auditory entrainment. In some embodiments, the neurostimulation database 118 may modulate specific neural oscillations, such as theta, alpha, beta, gamma, etc. associated with cognitive, motor, and emotional functions, enabling treatments for conditions such as neurodegenerative diseases, cognitive decline, and motor impairments. The neurostimulation database 118 provides the delivery of stimulation protocols through electrodes, displays, and speakers embedded in the headset 128. In some embodiments, for electrical stimulation, the neurostimulation database 118 may provide low-intensity electrical currents, which are delivered via surface electrodes 134 placed on the scalp or other regions, such as peripheral nerves. These currents can be modulated at precise frequencies and amplitudes to target specific neural circuits. In some embodiments, for sensory stimulation, the neurostimulation database 118 may coordinate visual stimuli, such as flickering lights or objects displayed on the VR screen 130, and auditory stimuli, such as amplitude-modulated sound waves, at therapeutic frequencies like 4 Hz, theta, or 40 Hz, gamma, to induce neural entrainment. In some embodiments, the system may also support multi-modal stimulation, combining electrical and sensory inputs to achieve synergistic effects, such as integrating tACS with synchronized visual and auditory stimuli for comprehensive neural modulation. In some embodiments, the neurostimulation database 118 may provide predefined or user-specific stimulation protocols. In some embodiments, the neurostimulation database 118 may contain safety measures, such as automatic shutdowns if abnormal neural activity, like epileptic patterns, is detected. In some embodiments, the neurostimulation database 118 also incorporates visual gratings, such as striped patterns, to induce narrow gamma oscillations in the visual cortex (V1). The gratings, adjusted for spatial frequency, contrast, orientation, and movement, may be used to both monitor disease progression and deliver therapeutic stimulation. In some embodiments, the neurostimulation database 118 may include visual patterns such as gratings (e.g., striped patterns) configured with specified spatial frequency, contrast, orientation, or motion parameters for delivery as part of a stimulation protocol. For patients with Parkinson's disease who show excessive beta activity and insufficient gamma activity in motor tasks, the neurostimulation database 118 may provide protocols for reducing rigidity and enhancing motor coordination. For example, the system may use beta power-reducing neurofeedback combined with gamma-flickering stimuli during hand movement tasks. In these tasks, patients engage in activities like squeezing a glowing, flickering ball synchronized to gamma frequencies, reinforcing motor cortex gamma oscillations, and improving fluidity of motion. In some embodiments, multisensory convergence may be utilized by combining visual, auditory, and proprioceptive feedback to achieve coordinated motor execution. In some embodiments, the stimulation protocol may prescribe stimulation frequencies, intensities, or phases to align with the patient's brainwave state associated with a given symptom or condition. For example, visual gratings and alternating auditory tones are synchronized to the patient's theta or gamma oscillations for tasks requiring memory recall or motor function. In some embodiments, the neurostimulation database 118 may enhance patient engagement and adherence by incorporating gamification elements. In some embodiments, the cultural and demographic context of the patient may be used to customize the VR environment. For example, a patient in Vermont may be shown a snowy mountain environment while a patient in Florida may be shown a sunny beach environment. In some embodiments, tasks may reward patients for maintaining neural activity within therapeutic ranges, such as keeping a beta power bar below a threshold or focusing on a theta-flickering object. In some embodiments, the neurostimulation database 118 may support combination therapies. For example, in managing L-Dopa-induced dyskinesia and impulsivity, the system may deliver gamma protocols to reduce involuntary movements and theta protocols to improve impulse control. Further, embodiments may include a cognitive task database 120, which may contain an array of cognitive tasks aimed at assessing, improving, and monitoring cognitive functions in users. The cognitive task database 120 may contain structured tasks that target areas such as memory, attention, problem-solving, emotional processing and motor coordination. The cognitive task database 120 may provide tasks categorized by difficulty level, domain, and therapeutic goal. In some embodiments, task parameters may be selected or configured prior to session execution based on session settings or predefined session structure. In some embodiments, the cognitive task database 120 may contain pre-designed cognitive tasks categorized by difficulty level, cognitive domain, such as working memory, episodic memory, attention, emotional processing, etc. and therapeutic goals. In some embodiments, the tasks may be implemented in modular formats, allowing seamless integration with the VR system's sensory and feedback mechanisms. In some embodiments, the cognitive task database 120 may support both active and passive tasks, ranging from gamified challenges, such as recall and recognition games, to structured assessments, such as free recall, spatial memory mapping, pattern recognition, or affective evaluation, etc. In some embodiments, the cognitive task database 120 may store a plurality of cognitive tasks that differ in structure, content, difficulty level, pacing, or presentation style. One or more tasks may be selected for a session based on predefined session parameters, therapeutic objectives, or user profile information determined prior to session execution. In some embodiments, cognitive tasks may be categorized or tagged according to functional domain, task complexity, emotional valence, or therapeutic context, enabling selection of tasks appropriate for a given session mode. Task parameters remain fixed during execution of the session. In some embodiments, the cognitive task database 120 may support incorporation of personalized content, allowing caregivers or users to associate familiar elements, such as personal photographs, known environments, text, or audio recordings, with cognitive tasks to enhance familiarity, emotional resonance, and engagement.

Further, embodiments may include a patient database 122, which contains patient-specific information that may indicate the stimulation protocols and VR scenarios presented to given patients, and the responses of these patients to those stimulation protocols and VR scenarios. The stored responses can include physiological activity measurements as recorded by the sensors 136 and associated session records. In some embodiments, the patient-specific data may also include patient-specific IDs and time stamps. In some embodiments, the patient database 124 may be accessible via a user interface of the system, and/or remotely by medical teams. In some embodiments, access by medical teams may provide the teams the ability to personalize therapeutic protocols, such as the nature of an initial stimulation protocol that is to be applied for the next session of a given patient. In some embodiments, the system may also include one or more software modules that transmit, such as using encrypted transmission, the patient-specific information to a remote server or database via a computer network allowing medical team access to the patient-specific information.

Further, embodiments may include a media database 124, which may store, organize, and manage personalized multimedia content, such as patient-specific images, videos, and audio files, for integration into the immersive VR environments. The media database 124 may facilitate the inclusion of emotionally and cognitively resonant content tailored to individual users to enhance engagement and therapeutic outcomes. The media database 124 may be designed to manage multimedia content that enhances the personalization of VR environments. The media database 124 may contain various types of digital content, including high-resolution images, 3D models, audio clips, and video files, and may include patient-uploaded materials, such as family photographs, recordings of personal locations, or other memory-related media. In some embodiments, the media database 124 may support multiple media formats to ensure compatibility with different input sources and rendering engines. In some embodiments, the media database 124 may integrate with other system components, such as the virtual reality database 116, cognitive task database 120, and neurostimulation database 118. The media database 124 provides dynamic retrieval and customization of media to align with therapeutic objectives. For example, personal photos uploaded via the user interface may be processed and synthesized into immersive VR scenarios using advanced rendering techniques. In some embodiments, the scenarios may be augmented with sensory stimuli, such as flickering lights or auditory cues, to enhance neural engagement. In some embodiments, the media database 124 may utilize metadata tagging for efficient organization and retrieval. For example, tags may include information about the type of media, patient relevance, such as “family photo” or “childhood memory”, and usage contexts, such as “relaxation therapy” or “memory recall task”. In some embodiments, machine learning algorithms may analyze the metadata to recommend or automatically select media for specific tasks or VR scenarios. In some embodiments, the media database 124 may support real-time integration with the headset system 128, transmitting selected media via a communication interface 106 to create an immersive, personalized VR experience. In some embodiments, the user's personalized media is displayed within a VR environment and synchronized with cognitive tasks and stimulation protocols to facilitate memory recall and therapeutic engagement.

Further, embodiments may include a cloud 126 which may be a network of remote servers that provide on-demand computing resources and services over the internet. The cloud 126 may consist of a collection of servers, storage devices, and networking equipment. Users may access the cloud 126 through a variety of devices, such as computers, smartphones, and tablets, using internet connectivity. In some embodiments, the architecture of the cloud 126 may be based on a distributed computing model, with multiple servers working together to provide services to users.

Further, embodiments may include a headset and system 128, which may be a wearable device designed to deliver therapeutic interventions through immersive virtual environments and sensory stimulation. The headset and system 128 may feature high-resolution, high-refresh-rate, such as 120 Hz or higher, LCD or OLED displays, ensuring crisp visuals and the capability to present flickering stimuli at specific frequencies, such as 40 Hz for gamma brainwave entrainment. The headset and system 128 may be integrated with advanced sensors 136 that measure a variety of physiological parameters, including brain activity via electroencephalogram (EEG) sensors, pupil dilation using pupillometry, and eye-tracking to monitor gaze direction and detect whether the eyes are open or closed. In some embodiments, additional sensors 136 may be integrated to measure heart rate, variability, and skin conductance to provide data to assess stress and engagement levels. In some embodiments, the headset and system 128 may include stereo or spatial audio speakers 132 that deliver synchronized auditory stimuli at therapeutic frequencies, complementing the visual inputs for multisensory entrainment. The headset and system 128 may be ergonomically designed with an adjustable, padded headband for comfortable extended use and feature a lightweight build to reduce fatigue. In some embodiments, an RGB LED indicator light may provide visual feedback on the device status, such as activation, pairing mode, or alerts, including medical emergencies. In some embodiments, power may be supplied via a rechargeable battery with optional direct plug-in or cradle charging support, enabling uninterrupted operation during sessions. In some embodiments, the headset and system 129 may communicate with the clarity network 102, including the virtual reality database 116, neurostimulation database 118, cognitive task database 120, and media database 124. The databases may include pre-configured visual and auditory stimulation patterns and immersive environments like cognitive games or calming landscapes, tailored to enhance engagement and therapeutic outcomes. In some embodiments, the headset and system 128 may communicate with the performance module 110 and recording module 112 which may collect, analyze and store physiological responses to the therapy delivery. In some embodiments, the system may support remote access for authorized caregivers or medical professionals to review stored session records and physiological data and to configure parameters for a subsequent session. In some embodiments, the frequencies at which sensory stimulations are provided may include, for example, gamma-corresponding frequencies in the 30-100 Hz range. In some embodiments, it may be required that the screen 130 of the headset and system 128 have an appropriately high enough refresh rate. In some embodiments, it may be required that the screen 130 have a refresh rate that is an integer multiple of those stimulation frequencies that are to be presented. For example, where the stimulation frequencies that are to be presented include a 40 Hz visual stimulus, given the Nyquist rule, 80 frames per second can be a minimum refresh rate for the screen of the headset and system 128. In some embodiments, a headset screen 130 refresh rate of 120 Hz or higher may be used. For example, a headset screen 130 refresh rate of 160 Hz, 200 Hz, or 240 Hz can provide for a stable delivery of 40 Hz visual stimulation.

Further, embodiments may include a screen 130, which may be a high-performance display panel embedded within the headset 128 that is responsible for presenting immersive virtual environments and delivering precise visual stimuli for therapeutic interventions. In some embodiments, the screen 130 may be remotely monitored, either at intervals or continuously, to ensure stimulation stability in order to dynamically adapt the rendering, frames per second, or battery expenditure, etc., in order to prioritise flicker stability. The screen 130 may be designed to support dynamic content delivery with high refresh rates, resolution, and brightness, enabling both passive and active engagement with the virtual reality scenarios and sensory stimulation protocols. In some embodiments, the screen 130 may be a high-definition, HD, ultra-high-definition, UHD, LCD, or OLED display optimized for therapeutic use. In some embodiments, the screen 130 may feature refresh rates of 120 Hz or higher to accurately present flickering visual stimuli at target frequencies, such as 40 Hz for gamma brainwave entrainment, while maintaining visual stability and avoiding latency. The resolution, such as 4K or higher, may provide visuals that are sharp, reducing eye strain and improving immersion. In some embodiments, the screens 130 brightness and color accuracy may be adjustable to support a wide range of therapeutic scenarios, from calming, low-light environments to more vivid, high-contrast tasks. In some embodiments, the screen 130 may deliver full-environment modulation, where the entire display flickers at a specific frequency, or localized stimulation through modulated objects or frames within the VR environment. In some embodiments, advanced rendering techniques may allow sinusoidal or square-wave luminance modulation for precise visual flicker delivery. In some embodiments, the system's ability to synchronize visual stimuli with auditory inputs further enhances multisensory neural entrainment, with phase-locking functionality ensuring alignment between visual and auditory frequencies. In some embodiments, the screen 130 refresh rate and modulation patterns may be precisely controlled to minimize visual artifacts, such as flickering instability. In some embodiments, integrated software may dynamically adjust screen 130 parameters, such as frequency, brightness, and color intensity, in response to patient-specific requirements. In some embodiments, the screen 130 may be used for displaying active cognitive and functional tasks. In some embodiments, the screen 130 may display gamified tasks, visual memory tests, or familiar environments, such as personalized photos or landscapes, making the therapy more engaging and personalized.

Further, embodiments may include speakers 132, which may be integrated audio output devices that provide high-quality sound, including therapeutic auditory stimuli, as part of the sensory stimulation protocols. In some embodiments, the speakers 132 may be positioned near the user's ears and may be designed to produce sound with a wide frequency range to provide clarity and precision across various auditory inputs. In some embodiments, the speakers 132 may be optimized for therapeutic purposes, featuring low distortion and high fidelity to preserve the integrity of the auditory signals. The speakers 132 may utilize bone conduction. In some embodiments, the system may dynamically synchronize the auditory and visual stimuli to maintain precise phase alignment to enhance neural entrainment and therapeutic efficacy. For example, auditory stimuli at 40 Hz can be phase-locked with visual flickers at the same frequency or complementary frequencies, allowing the system to target specific neural pathways effectively. In some embodiments, the speakers 132 may provide task-related audio guidance and feedback, such as instructions, questions, or results during cognitive and functional activities. In some embodiments, the speakers 132 may create immersive soundscapes by reproducing environmental sounds, music, or personalized audio elements to complement the virtual reality environment. In some embodiments, audio stimuli may be delivered according to a predefined stimulation protocol and synchronized with visual stimulation where applicable.

Further, embodiments may include electrodes 134, which may be specialized components designed to monitor and, in some cases, modulate the user's neural and physiological activity. The electrodes 134 may be used to collect electroencephalogram (EEG) data indicative of brain activity, including neural oscillations in one or more frequency bands such as theta, alpha, beta, and gamma. In some embodiments, the collected EEG data may be time-stamped and stored for subsequent review, reporting, or analysis in connection with one or more therapeutic sessions. In some embodiments, the electrodes 134 may be non-invasive and embedded within the headset 128 to maintain user comfort during extended sessions. In some embodiments, the electrodes 134 may be positioned to ensure optimal contact with the scalp, typically around the frontal, parietal, central, or occipital regions, depending on the specific neural signals being targeted. In some embodiments, the electrodes 134 may support peripheral nerve stimulation or deliver electrical signals for neuromodulation, such as transcranial direct current stimulation (tDCS) or transcranial alternating current stimulation (tACS), providing advanced therapeutic capabilities. In some embodiments, the electrodes 134 may be designed to minimize noise and maximize signal fidelity, employing high-precision amplification and filtering techniques to capture subtle electrical signals from the brain. In some embodiments, the collected data may include brainwave phase, amplitude, and frequency, which are processed in real time by the system's computational modules. In some embodiments, collected data may be stored for subsequent review or analysis outside the scope of session execution. In some embodiments, the electrodes 134 may deliver low-level electrical currents, modulated at specific frequencies, to target neural pathways associated with cognitive or motor functions. In some embodiments, the electrodes 134 may contribute to monitoring stress or abnormal neural activity, such as epileptic patterns, and may trigger safety mechanisms like stopping stimulation or alerting caregivers in critical situations.

Further, embodiments may include sensors 136, which may be designed to monitor a wide range of physiological and behavioral metrics, providing real-time data that enhances the therapeutic effectiveness of the system. In some embodiments, the sensors 136 may include EEG sensors to measure brain activity, pupillometry sensors to track pupil dilation, eye-tracking sensors to monitor gaze direction and eye openness, and heart rate sensors to evaluate cardiac activity and variability. In some embodiments, additional sensors 136 may measure skin conductance, respiratory rate, body temperature, and muscle activity via electromyography (EMG). In some embodiments, the EEG sensors 136 may be positioned to capture brainwave activity across different frequency bands, such as theta, alpha, beta, and gamma, which are associated with various cognitive and motor functions. In some embodiments, the pupillometry and eye-tracking sensors 136 may work in tandem to assess focus and attention by detecting gaze patterns and pupil responses to visual stimuli. In some embodiments, heart rate sensors 136 may provide insights into stress and relaxation states, while skin conductance sensors 136 may measure arousal levels through changes in sweat gland activity. In some embodiments, sensor data may be collected during session execution and stored for subsequent review or processing. In some embodiments, sensor data may support safety monitoring, including detecting conditions that trigger pausing or stopping a session in accordance with predefined safety procedures. Further, embodiments may include a communication interface 138, which may be a hardware or software component that enables communication between two or more electronic devices or systems. The communication interface 138 may include a set of protocols, rules, and standards that define how information is transmitted and received between the devices. The communication interface 138 may be a physical connector, wireless network, or software application. It may include components such as drivers, software libraries, and firmware that may be used to control and manage the communication process. In some embodiments, the communication interface 138 may be compatible with USB, Bluetooth, or Wi-Fi. The communication interface 138 may communicate with a network. Examples of networks may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN).

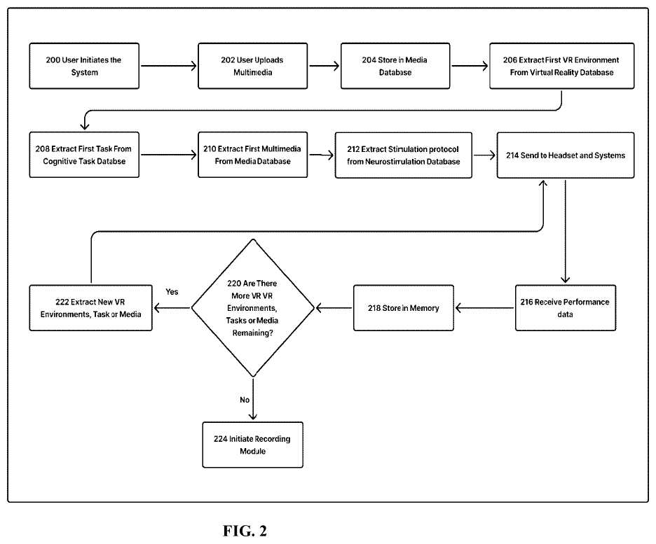

FIG. 2 illustrates the performance module 110. The process begins with the user initiating, at step 200, the system. In some embodiments, the user initiating or configuring a session may be the recipient of the therapy. In other embodiments, the user may be a caregiver, clinician, therapist, or other authorized member of a clinical or care team acting on behalf of the therapy recipient. In some embodiments, the performance module 110 may be activated by the user or through inputs made on the headset 128 interface. In some embodiments, the headset 128 may connect to the performance module 110, allowing the user to select session parameters that define a session mode corresponding to an intended therapeutic focus. By way of example, a session mode may be associated with memory-related engagement, attention-related engagement, emotional or affective engagement, motor engagement, or behavioral engagement. The user may also select one or more stimulation presentation settings for the session. The selected parameters may be transmitted to the performance module 110, which configures the session accordingly. In some embodiments, the performance module 110 may retrieve the necessary configuration settings, including database pointers for the virtual reality database 116, cognitive task database 120, neurostimulation database 118, and media database 124, to ensure they are ready for direct access. In some embodiments, the performance module 110 may also verify that the headset 128 sensors 136, such as EEG sensors, heart rate monitors, and eye-tracking devices, are functional and capable of collecting data during the session. The user uploads, at step 202, the personalized multimedia to the system. In some embodiments, the system allows the user or caregiver to upload personalized media, such as photographs, videos, or audio clips, through a secure interface. In some embodiments, the performance module 110 processes these files to ensure they meet system compatibility requirements, such as format and resolution. In some embodiments, advanced file-check algorithms may adjust or optimize the media for integration into VR environments. The performance module 110 stores, at step 204, the personalized multimedia in the media database 124. The uploaded personalized multimedia is indexed and stored in the media database 124. In some embodiments, metadata such as patient ID, media type, and context, such as family memories, favorite locations, etc., may be associated with each entry for efficient retrieval and integration during therapy sessions. The performance module 110 extracts, at step 206, the first VR environment from the virtual reality database 116. The performance module 110 retrieves the first VR environment from the virtual reality database 116. In some embodiments, the VR environment may be selected based on its alignment with therapeutic context or patient-specific requirements. In some embodiments, the VR environment may be based on data stored in the media database 124, such as a patient's former or current residence, personal location, including areas or places the patient has visited, etc. In some embodiments, parameters such as environmental visuals, audio settings, and immersive complexity are loaded for integration with the upcoming cognitive task. In some embodiments, the selection may be informed by patient history or pre-defined session configurations. The performance module 110 extracts, at step 208, the first task from the cognitive task database 120. The performance module 110 retrieves the first cognitive task from the cognitive task database 120. In some embodiments, the cognitive task may be designed to engage one or more functional domains, including cognitive, motor, emotional, affective, or behavioral domains, such as memory, attention, emotional processing, motor coordination, behavioral regulation, or problem-solving. In some embodiments, cognitive task parameters, including difficulty level, pacing, or interaction mechanics, may be aligned with the selected virtual-reality environment to create a cohesive and immersive experience, such that the task presentation supports engagement within the targeted functional domain during the therapeutic session. The performance module 110 extracts, at step 210, the first multimedia from the media database 124. The performance module 110 retrieves the first personalized multimedia entry from the media database 124. In some embodiments, the content is contextualized for integration into the selected VR environment and cognitive task. For example, a family photograph may be embedded into a memory recall task within a familiar VR setting. In some embodiments, the personalized multimedia may include images, videos, audio recordings, text, or other digital artifacts associated with the user's personal history, preferences, routines, or identity. Such multimedia may reflect familiar people, locations, activities, objects, or sensory experiences, and may be incorporated into the virtual-reality environment as visual elements, interactive objects, ambient context, or task-related stimuli. In some embodiments, the multimedia content may be selected to reflect aspects of the user's daily life, cultural background, habits, or personally meaningful experiences, thereby increasing familiarity and relevance of the virtual-reality session. In some embodiments, the personalized multimedia may be associated with descriptive information or contextual attributes that characterize the content, such as temporal context, location context, activity context, or other user-associated descriptors. These attributes may support flexible integration of the multimedia into different virtual-reality environments or cognitive tasks during session execution. At step 212, the performance module 110 configures delivery of a selected stimulation protocol in conjunction with the virtual-reality environment, cognitive task, and personalized multimedia selected for the session by selecting a simulation protocol from the neurostimulation database. The stimulation protocol may define one or more visual, auditory, or multisensory stimulation modalities and associated technical parameters, including stimulation frequency, temporal pattern, spatial pattern, intensity, contrast, or phase relationship. The stimulation may be embedded within, overlaid on, or synchronized with elements of the virtual-reality environment or personalized media presented to the user. In some embodiments, visual stimulation may be incorporated into the virtual-reality environment by modulating visual elements, textures, objects, or overlays according to predefined temporal or spatial patterns. For example, visual patterns, flickering elements, or luminance-modulated objects may be embedded within scenes that incorporate personalized images, videos, text, or familiar locations. In some embodiments, auditory stimulation may be incorporated through modulated sounds, tones, music, or voice recordings associated with personalized multimedia or environmental context presented during the session. In some embodiments, personalized multimedia, such as photographs, videos, audio recordings, or text associated with the user's personal history, may serve as carriers or contextual anchors for stimulation delivery. Visual stimulation patterns may be overlaid on or integrated into personalized images or videos, and auditory stimulation may be synchronized with familiar sounds or spoken content presented within the virtual-reality environment. The integration of stimulation with personalized and ecologically valid content may enhance relevance and familiarity while preserving a unified immersive experience.

As means of example, in some embodiments where the system is configured to memory-recall mode, the VR environment incorporates personalized multimedia such as family photographs, videos, or familiar locations uploaded by the patient. In such example, visual stimulation, such as gratings or flickering elements, may be overlaid on the multimedia content-such as a family photograph which may feature flickering patterns at 40 Hz, as part of a memory-related therapeutic session, thereby improving the ecological validity of neuromodulation delivery. The patient may engage with the multimedia content in an interactive three-dimensional setting, such as navigating a virtual gallery of personal memories, in which 40 Hz visual flicker or visual gratings are incorporated into the environment as part of a predefined stimulation protocol, providing neuromodulation delivery in a realistic and contextually meaningful manner. In some embodiments, auditory and visual stimuli may be combined to enhance engagement. For example, while viewing a virtual recreation of their childhood home, the system may play familiar sounds, such as a family member's voice or a song associated with the uploaded multimedia. In some embodiments, the sounds may be modulated to 40 Hz and synchronized with visual flickers in the environment, promoting gamma entrainment and enhancing memory consolidation. The patient may interact with objects in the VR scene, such as touching a virtual photograph to trigger associated audio, further linking memory retrieval with sensory cues. For semantic processing tasks related to memory recall, the VR environment may display uploaded text content, such as a favorite poem or a meaningful phrase, paired with theta-frequency flickers in alternating colors. This content may appear in a visually immersive space, such as floating text over a serene environment, to minimize distractions and focus the patient's cognitive efforts. Simultaneously, synchronized auditory cues, such as spoken words or low-frequency tones, enhance theta oscillations, aiding in semantic memory processing. In some embodiments, visual stimulation may use dynamic gratings, such as moving patterns or rotating stripes, integrated into the VR environment. For example, an uploaded video of a family gathering could include subtle overlays of moving gratings that flicker at target frequencies, promoting neural engagement while preserving the emotional and personal context of the multimedia content. In some embodiments, stimulation protocols having specific technical parameters may be pre-associated with a session mode or functional domain targeted by the session, such as memory-related engagement, attention-related engagement, emotional or affective engagement, motor engagement, or behavioral engagement. In some embodiments, multiple stimulation modalities may be combined within a session, delivered concurrently or sequentially in association with the selected virtual-reality environment and cognitive task. The configuration of stimulation delivery is determined during session assembly by the performance module 110 and remains fixed during session execution, without modification based on real-time physiological interpretation. The performance module 110 sends, at step 214, the VR environment, cognitive task, and personalized media to the headset and system 128. The selected VR environment, cognitive task, and personalized media are transmitted to the headset 128 for display and interaction through the cloud 126 via the communication interface 106. For example, in memory recall mode, the VR environment may incorporate personal multimedia, such as family photos or familiar locations, integrated into interactive tasks to enhance engagement and emotional connection. Cognitive tasks may include activities such as recognizing faces paired with names, navigating virtual representations of familiar environments, or recalling key details from displayed imagery. The multimedia integration allows the tasks to resonate with the patient's personal memories, fostering deeper engagement and potentially enhancing neural plasticity. In some embodiments, physiological signals, such as electroencephalogram activity, heart rate variability, or eye-tracking data, may be collected by sensors embedded in the headset system during session execution and stored for subsequent review or processing. Such information may be used, outside the scope of the ongoing session, to support evaluation of session content or to inform configuration of future sessions, without modifying delivery of the virtual-reality environment, tasks, or stimulation during the session.

The performance module 110 receives, at step 216, performance-related data from the headset system 128. In some embodiments, the headset system transmits data to the performance module 110 during session execution, including physiological signals, such as electroencephalogram data reflecting brainwave activity, heart rate variability, or eye-tracking information, as well as task-related information, such as task completion status, response timing, or interaction events. The received data may be associated with the corresponding session and stored for subsequent processing, review, or analysis outside the scope of the ongoing session. In some embodiments, the performance module 110 may receive indications from the headset system relating to operational or safety conditions during session execution. Such indications may be used to pause or terminate a session in accordance with predefined safety procedures, without evaluating therapeutic outcomes or modifying session content based on physiological interpretation. The performance module 110 stores, at step 218, the received performance-related and physiological data in memory 114. The stored data may be made available to the recording module 112 for subsequent analysis, review, or reporting. In some embodiments, the performance module 110 may perform data handling operations, such as formatting, time-stamping, or integrity checks, to support reliable storage of the collected data. The performance module 110 determines, at step 220, whether additional virtual-reality environments, cognitive tasks, or multimedia elements remain available in the virtual reality database 116, cognitive task database 120, or media database 124. If additional elements are available, the performance module 110 coordinates a transition to the next virtual-reality environment, cognitive task, or multimedia element in accordance with the predefined session structure, allowing for orderly progression through the session content. If it is determined that there are more VR environments and tasks remaining, the performance module 110 extracts, at step 222, the next VR environment from the virtual reality database 116, a task from the cognitive task database 120, and media from the media database 124 and the process returns to sending the VR environment, task, and media to the headset and system 128. In some embodiments, the session proceeds through the available virtual-reality environments, cognitive tasks, and multimedia elements in accordance with a predefined session structure associated with the selected session mode. If it is determined that no additional virtual-reality environments, cognitive tasks, or multimedia elements remain, the performance module 110 initiates, at step 224, the recording module 112, and the process returns to a session initiation state.

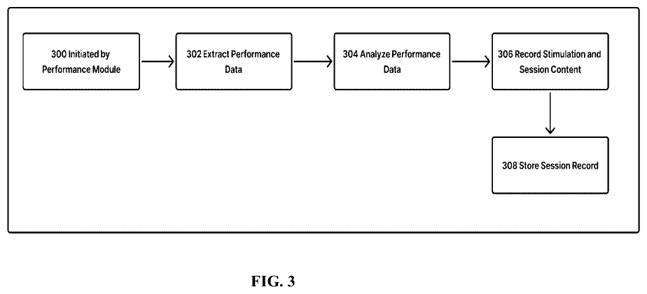

FIG. 3 illustrates the recording module 112. The process begins with the recording module 112 being initiated at step 300 by the performance module 110. The recording module 112 extracts, at step 302, the performance data from memory 114. The recording module 112 retrieves performance-related and physiological data from memory 114. In some embodiments, the recording module 112 retrieves data that was collected by the headset system 128 during execution of a session. In some embodiments, the data may include cognitive task metrics, such as task completion status or response timing, as well as physiological signals, such as electroencephalogram activity, heart rate variability, or pupillometric data acquired by sensors 136. The data may be organized or structured to support storage, review, or subsequent processing outside the scope of session execution. The recording module 112 processes, at step 304, the retrieved performance-related and physiological data.

The recording module 112 associates, at step 306, information identifying one or more stimulation protocols applied during the session with information identifying the virtual-reality environment, cognitive task, and personalized multimedia presented during the session. In some embodiments, the association reflects stimulation parameters that were selected during session assembly by the performance module 110 and delivered during session execution. The association supports creation of a session record documenting how stimulation, virtual-reality content, and multimedia elements were combined during the session, without initiating or modifying stimulation delivery or virtual-reality presentation. The recording module 112 stores, at step 308, information associated with the session record in one or more data repositories, such as memory 114 or a patient database 122. In some embodiments, the stored session record may include identifiers of the virtual-reality environment, cognitive task, personalized multimedia, and stimulation protocol used during the session, as well as associated performance-related or physiological data collected during session execution. The stored information may support subsequent review, reporting, or analysis by an authorized user outside the scope of the session. The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. A method for enhancing ecological validity of neurostimulation therapy, the method comprising:
receiving, via a communication interface, experiential data associated with a user, wherein the experiential data includes at least one of personalized media;
generating one or more virtual environments based on the experiential data, wherein the virtual environments include elements that replicate real-world environment based on one or more objects in the experiential data; and
providing, via a device-based stimulation system, a therapeutic protocol, wherein the therapeutic protocol integrates the virtual environments with one or more sensory neurostimulations.

2. The method of claim 1, wherein the device-based stimulation system includes a virtual reality headset.

3. The method of claim 1, wherein the experiential data includes location history of the user.

4. The method of claim 1, wherein the one or more virtual environments replicate real-world environment familiar to the user based on one or more objects in the experiential data.

5. The method of claim 1, wherein the virtual environments are further customized based on condition of the user.

6. The method of claim 1, wherein the sensory neurostimulations are provided at frequencies corresponding to neural oscillation.

7. The method of claim 1, wherein the sensory neurostimulations include visual and auditory stimuli, further comprising synchronizing the visual and auditory stimuli.

8. The method of claim 1, wherein the experiential data are received from one or more external sources associated with the user.

9. The method of claim 1, further comprising updating the therapeutic protocol based on user preference to the provided therapeutic protocol.

10. The method of claim 9, wherein the therapeutic protocol is updated based on sensor data associated with the user, wherein the sensor data is indicative of user engagement.

11. A system for enhancing ecological validity of neurostimulation therapy, the system comprising:
memory; and
a processor that executes instructions story in memory, wherein the processor executes instructions to:
receive, via a communication interface, experiential data associated with a user, wherein the experiential data includes at least one of personalized media;
generate one or more virtual environments based on the experiential data, wherein the virtual environments include elements that replicate real-world environment based on one or more objects in the experiential data; and
provide, via a device-based stimulation system, a therapeutic protocol, wherein the therapeutic protocol integrates the virtual environments with one or more sensory neurostimulations.

12. The system of claim 11, wherein the device-based stimulation system includes a virtual reality headset.

13. The system of claim 11, wherein the experiential data includes location history of the user.

14. The system of claim 11, wherein the one or more virtual environments replicate real-world environment familiar to the user based on one or more objects in the experiential data.

15. The system of claim 11, wherein the virtual environments are further customized based on condition of the user.

16. The system of claim 11, wherein the sensory neurostimulations are provided at frequencies corresponding to neural oscillation.

17. The system of claim 11, wherein the sensory neurostimulations include visual and auditory stimuli, further comprising synchronizing the visual and auditory stimuli.

18. The system of claim 11, wherein the experiential data are received from one or more external sources associated with the user.

19. The system of claim 11, wherein the processor executes further instructions to update the therapeutic protocol based on user preference to the provided therapeutic protocol.

20. A non-transitory, computer-readable storage medium, having embodied thereon a program executable by a processor to perform a method for enhancing ecological validity of neurostimulation therapy, the method comprising:
receiving, via a communication interface, experiential data associated with a user, wherein the experiential data includes at least one of personalized media;
generating one or more virtual environments based on the experiential data, wherein the virtual environments include elements that replicate real-world environment based on one or more objects in the experiential data; and
providing, via a device-based stimulation system, a therapeutic protocol, wherein the therapeutic protocol integrates the virtual environments with one or more sensory neurostimulations.
